(19) United States
(12) Patent Application Publication
(10) Pub. No.: US 2026/0360552 A1
(43) Pub. Date: Nov. 12, 2026

(54) VISION SCREENING DEVICE ENHANCEMENTS

(71) Applicant: Welch Allyn, Inc., Skaneateles Falls, NY (US)

(72) Inventor: David L. Kellner, Baldwinsville, NY (US)

(21) Appl. No.: 19/335,788

(22) Filed: Sep. 22, 2025

Related U.S. Application Data
(60) Provisional application No. 63/802,055, filed on May 8, 2025.

(51) Int. Cl. G16H 50/30; A61B 3/103; G06V 40/18; G16H 20/00

(52) U.S. Cl. CPC G16H 50/30; A61B 3/1035; G06V 40/18; G16H 20/00

(57) ABSTRACT

The present disclosure provides a device configured to receive user profile data including a patient's age, emit infrared light toward the patient's eyes, receive reflected light from the eyes, generate reflection data, and determine a degree of myopia, a presence of astigmatism, and an axis position of astigmatism. The device may predict, using a trained machine learning model, a likelihood of the patient developing high myopia based on the age, myopia degree, astigmatism presence, and axis position. The device may generate a user interface indicating likely high myopia development if the likelihood satisfies a threshold.

### RELATED APPLICATIONS

This application claims priority to, and the benefit of, U.S. Provisional Patent Application No. 63/802,055 entitled “VISION SCREENING DEVICE ENHANCEMENTS,” filed May 8, 2025, the contents of which are incorporated herein by reference in their entirety.

### TECHNICAL FIELD

This present disclosure is related to improvements to vision screening devices and associated systems to help detect highly myopic patients earlier in life. Doing so may enable optometrists and/or other healthcare professions to provide early intervention treatments to decrease the degree of myopia experienced by patients and to mitigate the chances of a patient developing more severe conditions such as retinal detachment, retinal tearing, macular degeneration, glaucoma, and/or cataracts.

### BACKGROUND

Vision screening devices may be utilized to determine if a patient is potentially near-sighted or far-sighted. However, other, potentially more problematic optometry issues may exist for given patients, and thus enhancements to vision screening devices would be helpful.

### SUMMARY OF THE INVENTION

The described innovations include a vision screening device that comprises one or more light emitters, one or more cameras, one or more processors, and non-transitory computer-readable media storing instructions that, when executed by the one or more processors, cause the vision screening device to perform several operations. Those operations may include receiving user profile data associated with a patient, the user profile data including an age of the patient at a time when vision screening is to occur. The operations may also include emitting, utilizing the one or more light emitters, at least infrared light in a direction of eyes of the patient, and receiving, utilizing the one or more cameras, reflected light from the eyes of the patient, the reflected light corresponding to the at least infrared light reflected from the eyes of the patient. The operations may also include generating, based at least in part on the reflected light, reflection data representing a processed version of the reflected light, the reflection data configured to be utilized by the one or more processors. The operations may also include determining, utilizing the reflection data, a degree of myopia of the patient, determining, utilizing the reflection data, a degree of astigmatism of the patient, and determining, utilizing the reflection data, an axis position of astigmatism of the patient. The operations may also include predicting, utilizing a trained machine learning model, a likelihood that the patient will have myopia with a diopter reading of at least −6.00, wherein the trained machine learning model utilizes at least the age of the patient, the degree of myopia, the degree of astigmatism, and the axis position of astigmatism to predict the likelihood. In response to the likelihood satisfying a threshold likelihood, the operations may include generating a user interface with an indication that the patient is likely to develop high myopia.

Additionally, the described innovations include a method comprising receiving user profile data associated with a patient, the user profile data including an age of the patient. The method may also include receiving reflected light from eyes of the patient, the reflected light corresponding to at least infrared light emitted in a direction of the eyes of the patient. The method may also include generating, based at least in part on the reflected light, reflection data representing a processed version of the reflected light. The method may also include determining, utilizing the reflection data, a degree of myopia of the patient and determining, utilizing the reflection data, a degree of astigmatism of the patient. The method may also include predicting, utilizing a trained model, a likelihood that the patient will have myopia with a diopter reading of at least −6.00, wherein the trained model utilizes at least the age of the patient, the degree of myopia, the degree of astigmatism, and/or a location of the astigmatism to predict the likelihood. Based at least in part on the likelihood satisfying a threshold likelihood, the method may also include generating a user interface with an indication that the patient is likely to develop high myopia.

Furthermore, the described innovations include a system comprising one or more processors and non-transitory computer-readable media storing instructions that, when executed by the one or more processors, cause the system to perform several operations. The operations may include receiving data indicating an age of a patient and generating, based at least in part on reflected light from eyes of the patient, reflection data representing a processed version of the reflected light. The operations may also include determining, utilizing the reflection data, a degree of myopia of the patient and determining, utilizing the reflection data, a degree of astigmatism of the patient. The operations may also include predicting, utilizing a trained model, a likelihood that the patient will have myopia with a diopter reading of at least −6.00, wherein the trained model utilizes at least the age of the patient, the degree of myopia, and the degree of astigmatism to predict the likelihood. The operations may also include generating a user interface with an indication that the patient is likely to develop high myopia.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an example environment for vision screening device enhancements, according to aspects of the present disclosure.

FIG. 2 illustrates an example vision screening device and an associated graphical user interface showing an indication of predicted high myopia, according to aspects of the present disclosure.

FIG. 3 illustrates a conceptual diagram of example data and components utilized for enhancing a vision screening device, according to aspects of the present disclose.

FIG. 4A illustrates a graph associating axial length growth rate with astigmatism, according to aspects of the present disclose.

FIG. 4B illustrates a graph associating axial length growth rate, astigmatism, and hyperopia, according to aspects of the present disclose.

FIG. 4C illustrates a graph associating axial length growth rate and axis position of astigmatism, according to aspects of the present disclose.

FIG. 5 illustrates a flowchart of a process for retrieving several vision-related metrics and user-specific metrics to predict whether a patient will have high myopia at a later time, according to aspects of the present disclose.

FIG. 6 illustrates a flowchart of a process for use of a vision screening device to predict high myopia at a later time, according to aspects of the present disclose.

FIG. 7 illustrates a flowchart of a process for use of a trained machine learning model to predict high myopia at a later time, according to aspects of the present disclose.

FIG. 8 illustrates a flowchart of a process for implementing vision screening device enhancements, according to an embodiment.

FIG. 9 illustrates a flowchart of another process for implementing vision screening device enhancements, according to aspects of the present disclosure.

FIG. 10 illustrates a flowchart of a method for generating and utilizing machine learning models, according to an embodiment.

### DETAILED DESCRIPTION

The vision screening device described herein incorporates advanced light emission and detection capabilities to predict high myopia in a patient. The device may emit infrared light towards a patient's eyes and capture the reflected light using specialized cameras. This approach allows for non-invasive examination of the eye's structures and characteristics. The system described herein may process the captured reflected light to generate reflection data, which may serve as the basis for various eye measurements and assessments. This reflection data may be analyzed by the device's processors to determine key vision metrics, including the degree of myopia, degree of astigmatism, and the axis position of astigmatism in the patient's eyes.

The vision screening device may utilize such metrics along with other information as described herein to predict the likelihood of a patient developing high myopia in the future. This prediction may be made using a trained machine learning model that takes into account multiple factors, including the patient's age, current degree of myopia, degree of astigmatism, and axis position of astigmatism, for example. The model may analyze these inputs to estimate the probability of the patient reaching a myopia level of −6.00 diopters or greater (defined herein as “high myopia”). The device may incorporate a user interface that presents examination results and risk assessments in a clear, actionable format. When the predicted likelihood of high myopia exceeds or otherwise satisfies a certain threshold, the system may generate an alert on the user interface, indicating that the patient is at risk of developing high myopia. This feature enables healthcare providers to identify high-risk patients early and initiate appropriate interventions. Furthermore, the system may automate the referral process for high-risk patients. When a patient is identified as having a high likelihood of developing severe myopia, the device can generate a referral data object containing relevant examination results and images. This feature streamlines the process of connecting at-risk patients with appropriate specialists for further evaluation and treatment.

Another innovative aspect is the system's ability to recommend specific treatment options based on the examination results and risk assessment. The trained model may evaluate various potential treatments and suggest the most appropriate option for the individual patient. This personalized approach to treatment recommendation can help optimize patient care and outcomes.

The system also has the capability to perform additional eye examinations, such as a red reflex examination. The results of these supplementary tests can be incorporated into the machine learning model, further refining its predictive accuracy for high myopia risk assessment. Furthermore, the system may analyze physical characteristics of the patient, such as skin tone, iris color, and/or iris transparency. These factors may be determined from image data captured by the device's cameras and can be used by the machine learning model to enhance the accuracy of its predictions, potentially accounting for personal factors that may influence myopia progression. The system may also generate predictions for each of the patient's eyes individually, which may allow for a more comprehensive assessment, as doing so can account for potential differences in myopia progression between the left and right eyes of a patient.

The innovations also include methods for continually improving the accuracy of the predictive model. For example, the system may collect feedback data over time, comparing predicted outcomes with actual patient results. This data can be used to generate updated training datasets, allowing the machine learning model to be refined and improved over time.

The described vision screening innovations may provide several technical advantages to computer systems implementing the technology. By utilizing advanced light emission and detection techniques coupled with machine learning models, the system may achieve improved processing efficiency when analyzing eye examination data. The generation of processed reflection data may enhance data storage and retrieval by providing a standardized format for eye measurements that can be efficiently utilized by the system's processors. The incorporation of multiple data points such as age, degree of myopia, astigmatism metrics, and physical characteristics into the predictive model may increase the reliability and accuracy of high myopia risk assessments. The automated referral process and treatment recommendations may lead to better resource allocation within healthcare systems by streamlining patient management workflows. Additionally, the user interface enhancements for displaying examination results and risk assessments may improve interface responsiveness, allowing healthcare providers to quickly access and interpret critical patient information.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

Additional details are described below with reference to several example embodiments.

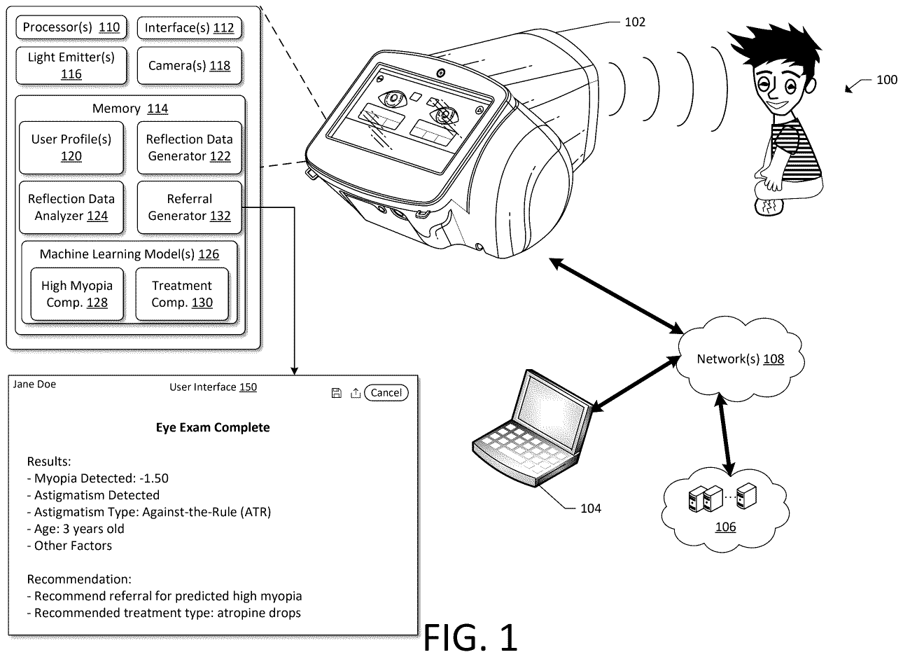

FIG. 1 illustrates an environment 100 for a vision screening system. The environment 100 may include a vision screening device 102, a computing device 104, a server 106, and a network 108.

The vision screening device 102 may comprise one or more light emitters, one or more cameras, one or more processors, and non-transitory computer-readable media. In some cases, the vision screening device 102 may utilize the one or more light emitters to emit light towards a patient's eyes during an examination. The one or more cameras of the vision screening device 102 may capture reflected light from the patient's eyes. The one or more processors and non-transitory computer-readable media of the vision screening device 102 may process and analyze the captured light data to perform vision assessments, as described in detail below.

The computing device 104 may be used to interface with the vision screening device 102. In some cases, the computing device 104 may display results from vision screenings performed by the vision screening device 102. The server 106 may store data related to vision screenings and may perform additional processing of screening results. The network 108 may facilitate communication between the vision screening device 102, the computing device 104, and the server 106. The system comprising the vision screening device 102, the computing device 104, and the server 106 may include one or more processors and non-transitory computer-readable media storing instructions. These instructions, when executed by the one or more processors, may cause the system to perform various operations related to vision screening and analysis.

The vision screening device 102 may include several internal components as illustrated in FIG. 1. The vision screening device 102 may comprise a processor 110, an interface 112, a memory 114, a light emitter 116, and a camera 118. In some cases, the processor 110 of the vision screening device 102 may control the operations of the other components and process data received from the camera 118. The interface 112 may enable communication between the vision screening device 102 and other devices in the environment 100, such as the computing device 104 or the server 106 via the network 108. The memory 114 of the vision screening device 102 may store instructions and data used by the processor 110. In some cases, the memory 114 may store reflection data generated from light captured by the camera 118.

The light emitter 116 of the vision screening device 102 may emit infrared light towards a patient's eyes during a vision screening examination. The camera 118 may receive reflected light from the patient's eyes, where the reflected light may correspond to the infrared light emitted by the light emitter 116. In some cases, the processor 110 may analyze the reflected light captured by the camera 118 to determine various vision metrics for the patient. The vision screening device 102 may utilize these components to perform vision assessments and generate predictions about a patient's likelihood of developing high myopia.

The memory 114 of the vision screening device 102 may store several software components that enable the vision screening device 102 to perform various operations related to vision screening and analysis. These software components may include a user profile 120, a reflection data generator 122, a reflection data analyzer 124, a machine learning model 126, a high myopia component 128, a treatment component 130, and a referral generator 132, for example.

The user profile 120 may serve as a comprehensive data storage and management component for patient-specific information within the vision screening device 102. This component may store a wide range of patient data, including but not limited to, demographic information such as name, date of birth, gender, and contact details. The user profile 120 may also maintain a record of the patient's medical history, particularly focusing on vision-related conditions, previous diagnoses, and treatments. In some implementations, the user profile 120 may store historical vision screening results, allowing for trend analysis and comparison over time. The user profile 120 may also include family medical history, especially related to vision conditions, which may be relevant for predicting myopia progression. Additionally, the user profile 120 may store lifestyle factors that could influence vision health, such as screen time habits, outdoor activity levels, and/or dietary information. The user profile 120 may be designed to integrate with other components of the vision screening device 102, providing relevant patient data to inform the analysis performed by the reflection data analyzer 124 and the machine learning model 126. In examples, the user profile 120 may be updated after individual screening sessions, ensuring that the most current patient information is available for subsequent assessments and predictions.

The reflection data generator 122 may be a sophisticated component of the vision screening device 102 designed to process and transform raw light data captured by the camera 118 into a standardized format suitable for further analysis. By way of example, the light emitter(s) 116 may be caused to emit light in the direction of a patient. That light may come into contact with the patient, and particularly the patient's eyes, and reflect back to the camera 118 where such raw light data may be received by the camera. This reflection data generator 122 may employ various signal processing techniques, which may include digital filtering, noise reduction algorithms, and/or image enhancement methods. The reflection data generator 122 may also incorporate calibration routines to account for variations in lighting conditions and/or individual camera characteristics. In examples, the reflection data generator 122 may utilize machine learning algorithms to identify and extract relevant features from the captured light data. The output of this component may be a structured data format that represents characteristics of the reflected light, potentially including intensity maps, spectral information, and/or derived metrics related to eye physiology. The reflection data generator 122 may be designed to operate in real-time, in examples, processing incoming light data as it is captured, or the reflection data generator 122 may perform batch processing on stored data sets. The generated reflection data may be optimized for efficient storage and retrieval, potentially using compression techniques that preserve essential information while reducing data volume in a way that enhances and improves the functioning of the vision screening device 102. This processed reflection data may serve as the foundation for subsequent analysis by other components of the vision screening device 102, such as the reflection data analyzer 124 and the machine learning model 126.

The reflection data analyzer 124 may be designed to interpret and extract information from the processed reflection data. In examples, the reflection data analyzer 124 may employ advanced image processing algorithms to identify specific features within the reflection data that correspond to various eye characteristics. These algorithms may include edge detection techniques to delineate eye structures, pattern recognition methods to identify specific reflection patterns associated with different refractive errors, and/or spectral analysis to extract information from different wavelengths of reflected light. The reflection data analyzer 124 may also incorporate machine learning models 126 trained on large datasets of eye reflection patterns, enabling it to recognize subtle indicators of various eye conditions that may not be immediately apparent.

In examples, the reflection data analyzer 124 may work in conjunction with other components of the vision screening device 102 to provide a comprehensive analysis of the patient's eye health. For example, the reflection data analyzer 124 may interface with the user profile 120 to consider patient-specific factors when interpreting reflection data, potentially adjusting its analysis based on age, medical history, or genetic predispositions. The reflection data analyzer 124 may also generate quantitative metrics that can be utilized by the machine learning model 126 for predicting myopia progression. These metrics may include measurements of corneal curvature, lens opacity, retinal reflectivity, presence of refractive errors, degree of refractive errors, presence of astigmatism, type of astigmatism, and the like. In examples, the reflection data analyzer 124 may be capable of analyzing data from multiple examinations over time, enabling detection of subtle changes in eye characteristics that may indicate the onset or progression of various eye conditions, including high myopia.

The machine learning model 126 may be designed to analyze and interpret complex eye-related data. For example, the machine learning model 126 may incorporate various algorithms such as convolutional neural networks, random forests, and/or gradient boosting machines tailored to process specific types of data generated during vision screenings. The machine learning model 126 may be trained on a large dataset of eye examination results, including reflection data, patient demographics, and/or historical progression of vision conditions. This training process may enable the model to identify subtle patterns and correlations that may not be apparent through traditional analysis methods.

In examples, the machine learning model 126 may utilize transfer learning techniques, allowing the machine learning model 126 to leverage knowledge gained from broader vision-related tasks to improve performance on specific myopia prediction tasks. The model may be designed with a modular architecture, potentially incorporating multiple sub-models specialized for different aspects of vision analysis, such as corneal topography interpretation or retinal image processing. This modular approach may allow for flexibility in updating or refining specific components of the model as new research or data becomes available. Examples of such sub-models may include the high myopia component 128 and/or the treatment component 130. The machine learning model 126 may also be capable of processing various types of input data, including numerical measurements from vision tests, image data from eye scans (such as from the reflection data analyzer 124, and/or textual information from patient histories (such as from the user profiles 120). In examples, the machine learning model 126 may employ feature extraction techniques to distill relevant information from potentially diverse input data, creating a unified representation for analysis. The model may also incorporate mechanisms for handling missing or noisy data, enhancing its robustness in real-world clinical settings. In examples, these mechanisms may include image stabilization to account for movement of the camera 118 and/or movement of the patient.

With respect to the sub-models, the high myopia component 128 may be structured as a specialized neural network within the machine learning model 126, designed to focus specifically on identifying patterns indicative of high myopia development. This component may incorporate multiple layers of convolutional and/or fully connected nodes, with each layer potentially optimized to detect specific features associated with severe myopia progression. The architecture of the high myopia component 128 may include skip connections and/or residual blocks to facilitate the flow of information across different levels of abstraction, allowing for more nuanced analysis of complex eye data patterns.

A function of the high myopia component 128 may be to analyze and interpret the various input metrics from the reflection data analyzer 124, with a specific focus on predicting the likelihood of a patient developing high myopia. This component may be designed to identify subtle correlations between different vision metrics, patient demographics, and/or historical data that may indicate an increased risk of severe myopia, particularly in patients of a young age at the time of vision screening. The high myopia component 128 may generate a probability score and/or risk assessment for high myopia development, which can be used to trigger alerts or recommendations for early intervention strategies, as described in more detail below.

The high myopia component 128 may work in conjunction with other elements of the vision screening device 102 to perform these functions. For example, the high myopia component 128 may receive preprocessed data from the reflection data analyzer 124, incorporating information from the user profile 120 to contextualize its analysis based on patient-specific factors. The output from the high myopia component 128 may be utilized by the treatment component 130 to inform personalized treatment recommendations. Additionally, the high myopia component 128 may interface with the referral generator 132, providing data to support the creation of detailed referral documents and/or data objects for patients identified as high-risk for severe myopia.

The high myopia component 128 may also be designed as a standalone component that can be updated and/or replaced as additional research on myopia progression becomes available. Alternative versions of the high myopia component 128 may incorporate different machine learning algorithms, such as recurrent neural networks for analyzing time-series data from multiple screenings, and/or ensemble methods combining predictions from multiple sub-models for improved accuracy. The implementation of the high myopia component 128 may involve a multi-stage process, including initial training on large datasets of historical vision screening results and subsequent fine-tuning based on ongoing feedback and performance metrics. The high myopia component 128 may be developed using machine learning frameworks, allowing for efficient deployment and execution on the vision screening device 102. Regular updates and recalibration of the high myopia component 128 may be performed to ensure its predictions remain accurate and relevant as new data is collected and analyzed across multiple patient populations and demographic groups.

By way of nonlimiting example, the reflection data analyzer 124 may determine a degree of myopia of the patient in question, and/or a degree of astigmatism of the patient, and/or an axis position of the astigmatism of the patient. Additionally, the age of the patient may be determined from the user profile 120. The high myopia component 128 may utilize this data to predict a likelihood that the patient will have myopia with a diopter reading of at least −6.00, indicating predicted high myopia of the patient. With respect to the axis position of astigmatism, the high myopia component 128 may be configured to determine whether the astigmatism has an axis position of with-the-rule or against-the-rule. When the axis position indicates the patient has with-the-rule astigmatism, the high myopia component 128 may bias such that predicting high myopia is less likely. When the axis position indicates that patient has against-the-rule astigmatism, the high myopia component 128 may bias such that predicting high myopia is more likely. Additionally, or alternatively, red reflex examination results may be determined and may be utilized by the high myopia component 128 to predict the likelihood of high myopia. In still further examples, the camera 118 may utilize image data corresponding to images of the patient to determine aspects such as skin tone of the patient, iris color of the patient, an iris transparency metric of the patient, and the like. Some of all of this information may be utilized by the high myopia component 128 to predict the likelihood that the patient will have high myopia. In still further examples, myopia-related information of a parent of the patient may be determined (such as from the user profile 120), and such parent information may be utilized by the high myopia component 128 to predict the likelihood of high myopia. Furthermore, predicting the likelihood of high myopia may be perform for each of the patient's eyes. For example, some of all of the data types described just above may be determined for each of the patient's eyes, and prediction as to the likelihood that the patient will have high myopia may be made individually for both eyes.

The treatment component 130 may be designed to analyze the output from the high myopia component 128 and other relevant data to generate personalized treatment recommendations for patients. This treatment component 130 may incorporate a decision-making algorithm that considers multiple factors, including the predicted likelihood of high myopia development, the patient's current vision metrics, age, medical history, and potential contraindications. The treatment component 130 may access a comprehensive database of available myopia management strategies, which may include optical interventions such as specialized contact lenses or eyeglasses, pharmaceutical treatments like low-dose atropine eye drops, and/or lifestyle modifications such as increased outdoor time or reduced screen time, amongst other treatments. In examples, the treatment component 130 may utilize a multi-criteria decision analysis framework to weigh the potential benefits and risks of different treatment options for individual patients. The treatment component 130 may also consider factors such as treatment efficacy, potential side effects, patient compliance likelihood, and/or cost-effectiveness when generating its recommendations. Additionally, the treatment component 130 may be designed to adapt its recommendations based at least in part on ongoing feedback and treatment outcomes, potentially incorporating a reinforcement learning approach to refine its decision-making process over time. The treatment component 130 may also generate visualizations and/or simulations of potential treatment outcomes to assist healthcare providers in explaining treatment options to patients and their families.

The referral generator 132 may be designed to automate and streamline the process of creating referrals for patients identified as high-risk for developing severe myopia. This component may integrate data from multiple sources within the system, including the high myopia component 128, the user profile 120, and/or the reflection data analyzer 124, to generate comprehensive and personalized referral documents and/or data objects. The referral generator 132 may incorporate natural language processing algorithms to translate complex medical data into actionable recommendations for healthcare providers. In some implementations, the referral generator 132 may include a prioritization component that assesses the urgency of referrals based on factors such as predicted myopia progression rate, patient age, and current vision metrics. The component may interface with external electronic health record systems to ensure transfer of referral information to appropriate specialists. Additionally, the referral generator 132 may feature a follow-up tracking system that monitors referral outcomes and provides feedback to improve future referral accuracy. The referral generator 132 may also have the capability to generate visual aids, such as progression charts or comparison images, to be included in referral packages, enhancing the communication of complex vision data to both healthcare providers and patients.

By way of example, the referral generator 132 may determine that a patient should be referred to a healthcare professional associated with high myopia based at least in part on the likelihood of developing high myopia satisfying a threshold likelihood. The referral generator 132 may generate a referral data object containing an indication of the patient's likelihood of developing high myopia, along with one or more images corresponding to reflected light from the patient's eyes and/or other aspects of a vision screening exam. Additionally, in examples, the referral generator 132 may automatically generate a referral that incorporates the referral data object. This approach may streamline the referral process for patients identified as having a high risk of developing severe myopia, potentially enabling earlier intervention and more effective management of the condition.

It should be noted that the exchange of data and/or information as described herein may be performed only in situations where a user has provided consent for the exchange of such information. For example, a user may be provided with the opportunity to opt in and/or opt out of data exchanges between devices and/or with the systems and/or for performance of the functionalities described herein. Additionally, when one of the devices is associated with a first user account and another of the devices is associated with a second user account, user consent may be obtained before performing some, any, or all of the operations and/or processes described herein.

As used herein, a processor, such as processor(s) 110, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 110 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 110 may possess its own local memory, which also may store program components, program data, and/or one or more operating systems.

The memory 114 may include volatile and nonvolatile memory, removable and non-removable media implemented in any method or technology for storage of information, such as computer-readable instructions, data structures, program component, or other data. Such memory 114 includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 114 may be implemented as computer-readable storage media (“CRSM”), which may be any available physical media accessible by the processor(s) 110 to execute instructions stored on the memory 114. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s).

Further, functional components may be stored in the respective memories, or the same functionality may alternatively be implemented in hardware, firmware, application specific integrated circuits, field programmable gate arrays, or as a system on a chip (SoC). In addition, while not illustrated, each respective memory, such as memory 114 discussed herein may include at least one operating system (OS) component that is configured to manage hardware resource devices such as the network interface(s), the I/O devices of the respective apparatuses, and so forth, and provide various services to applications or components executing on the processors. Such OS component may implement a variant of the FreeBSD operating system as promulgated by the FreeBSD Project; other UNIX or UNIX-like variants; a variation of the Linux operating system as promulgated by Linus Torvalds; the FireOS operating system from Amazon.com Inc. of Seattle, Washington, USA; the Windows operating system from Microsoft Corporation of Redmond, Washington, USA; LynxOS as promulgated by Lynx Software Technologies, Inc. of San Jose, California; Operating System Embedded (Enea OSE) as promulgated by ENEA AB of Sweden; and so forth.

The network interface(s) 112 may enable messages between the components and/or devices shown in environment 100 and/or with one or more other remote systems, as well as other networked devices. Such network interface(s) 112 may include one or more network interface controllers (NICs) or other types of transceiver devices to send and receive messages over the network.

For instance, each of the network interface(s) 112 may include a personal area network (PAN) component to enable messages over one or more short-range wireless message channels. For instance, the PAN component may enable messages compliant with at least one of the following standards IEEE 802.15.4 (ZigBee), IEEE 802.15.1 (Bluetooth), IEEE 802.11 (WiFi), or any other PAN message protocol. Furthermore, each of the network interface(s) 112 may include a wide area network (WAN) component to enable message over a wide area network.

In some instances, the server 106 or portions thereof may be local to an environment associated the vision screening device 102 and/or the computing device 104. In some instances, some or all of the functionality of the server 106 may be performed by the vision screening device 102 and/or the computing device 104. Also, while various components of the vision screening device 102 have been labeled and named in this disclosure and each component has been described as being configured to cause the processor(s) to perform certain operations, it should be understood that the described operations may be performed by some or all of the components such as the computing device 104 and/or the service 106, and/or other components not specifically illustrated.

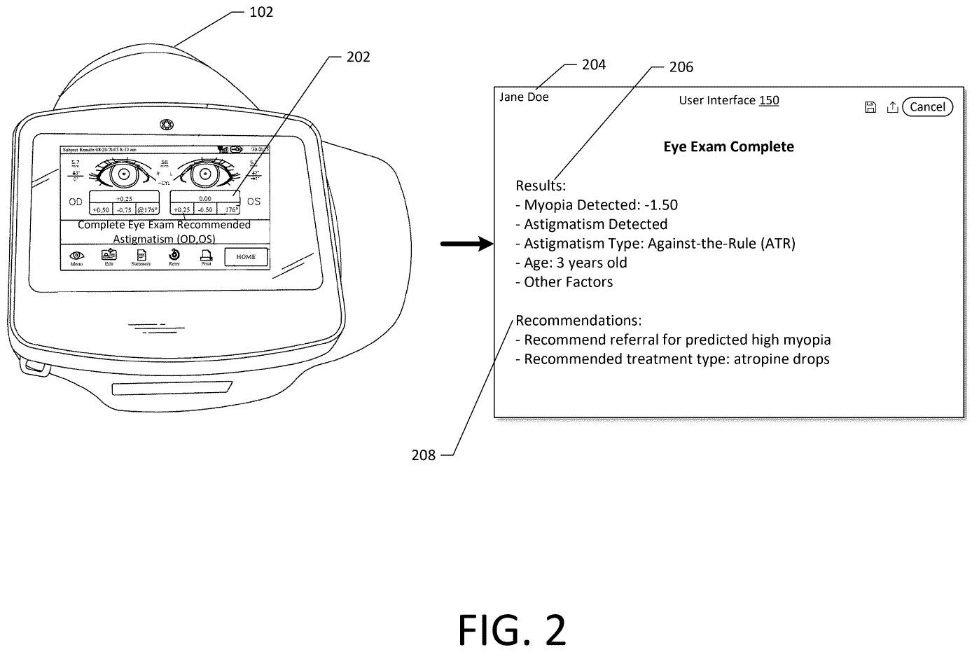

FIG. 2 illustrates an example vision screening device and an associated graphical user interface showing an indication of predicted high myopia, according to aspects of the present disclosure. The vision screening device 102 may generate a user interface 150 to display results and information from vision screenings. FIG. 2 illustrates an example of the user interface 150 that may be presented on the vision screening device 102 and/or the computing device 104.

The user interface 150 may include several sections to organize and present screening data. A vision metrics section 202 may display graphical representations of key eye examination measurements. In examples, the vision metrics section 202 may include visualizations of myopia and astigmatism metrics for each eye. The vision metrics section 202 may also present an axial length growth rate measurement, which may be useful for tracking potential myopia progression over time. The vision metrics section 202 may also include any other information that may be gathered during the course of a vision screening.

Once a vision screening is complete, a prediction of high myopia likelihood may be made as described in detail with respect to FIG. 1. A patient information section 204 may be positioned in the user interface 150. This patient information section 204 may display demographic data about the patient, potentially retrieved from the user profile 120 stored in the memory 114 of the vision screening device 102. The patient information section 204 may include details such as the patient's name, age, and date of examination.

Additionally, the user interface 150 may include a results section 206 that may present key findings from the vision screening. The results section 206 may include detected conditions such as the degree of myopia and astigmatism measurements for each eye. In examples, the results section 206 may also display the likelihood of the patient developing high myopia, as determined by the high myopia component 128 of the machine learning model 126. The user interface 150 may also include a recommendations section 208. This recommendations section 208 may provide suggested next steps based at least in part on the examination results and risk assessment. The recommendations section 208 may display treatment options generated by the treatment component 130 and/or referral information created by the referral generator 132.

The user interface 150 may be designed to present examination data and recommendations in an organized layout that enables efficient review of screening results in a time-sensitive manner. By consolidating information from various components of the vision screening device 102, the user interface 150 may facilitate clear communication of vision health status and potential risks to healthcare providers and patients.

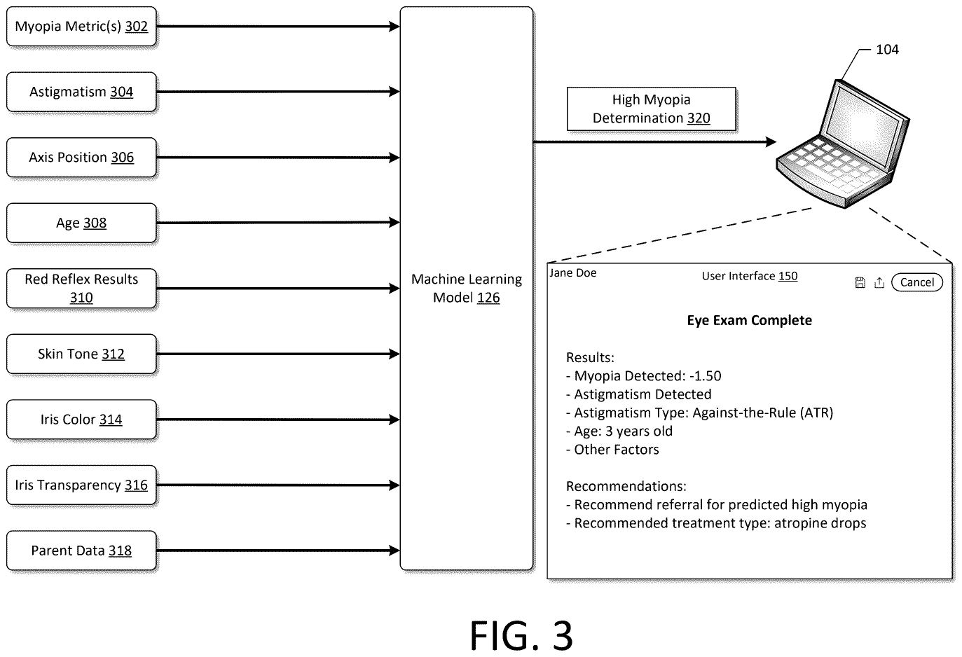

FIG. 3 illustrates a conceptual diagram of example data and components utilized for enhancing a vision screening device, according to aspects of the present disclose. FIG. 3 includes a number of nonlimiting data types and/or metrics that may be utilized by a machine learning model, such as machine learning model 126, to predict a likelihood that a given patient will experience high myopia.

To illustrate, The machine learning model 126 may receive multiple input metrics and generate output determinations. The machine learning model 126 may utilize various input metrics to predict the likelihood of a patient developing high myopia. These input metrics may include a myopia metric 302, an astigmatism metric 304, an axis position metric 306, and an age metric 308. The myopia metric 302 may represent the current degree of myopia in the patient's eyes. The astigmatism metric 304 may indicate the degree of astigmatism present. The axis position metric 306 may specify the orientation of the astigmatism. The age metric 308 may represent the patient's age at the time of the vision screening.

In examples, the machine learning model 126 may also incorporate a red reflex metric 310. The red reflex metric 310 may be derived from a red reflex examination performed by the vision screening device 102. This metric may provide additional information about the patient's eye health and structure as well as information about a demographic of the patient. The machine learning model 126 may also consider physical characteristics of the patient. These characteristics may include a skin tone metric 312, an iris color metric 314, and/or an iris transparency metric 316. The skin tone metric 312 may represent the patient's skin pigmentation. The iris color metric 314 may indicate the color of the patient's iris. The iris transparency metric 316 may provide information about the opacity or clarity of the patient's iris. In further examples, the machine learning model 126 may utilize parent data 318 as an input. The parent data 318 may include information about the vision health and history of the patient's parents, which may be relevant for predicting myopia progression.

The machine learning model 126 may calculate attributes impacting axial length growth based at least in part on age group, sphere/cylinder axis group, and patient-specific image group. These calculations may involve analyzing patterns and correlations within the input data to identify factors that may influence eye growth and myopia development. The machine learning model 126 may use both categorical predictors and continuous predictors as inputs. Categorical predictors may include discrete variables such as age groups or astigmatism types. Continuous predictors may include numerical measurements like the degree of myopia or astigmatism. By processing these various input metrics, the machine learning model 126 may generate predictions regarding the likelihood of a patient developing high myopia. These predictions may be utilized by the vision screening device 102 to provide early detection and intervention recommendations for patients at risk of severe myopia.

The machine learning model 126 may generate a high myopia determination 320 based at least in part on the analysis of the various input metrics. This high myopia determination 320 may represent a prediction of the likelihood that a patient will develop high myopia in the future. In examples, the machine learning model 126 may be trained to predict likelihoods of high myopia from a single vision screening session, allowing for early detection and intervention.

The high myopia determination 320 may be transmitted from the vision screening device 102 to the computing device 104 via the network 108, for example. The computing device 104 may then display the high myopia determination 320 through the user interface 150. In examples, the user interface 150 may present the high myopia determination 320 within the results section 206 from FIG. 2, providing healthcare professionals with a clear indication of the patient's risk for developing severe myopia. Additionally, the vision screening device 102 may determine and display a recommended treatment option using the trained machine learning model 126. A treatment component of the machine learning model 126 may analyze the high myopia determination 320 along with other relevant patient data to generate personalized treatment recommendations. These recommendations may be displayed in the recommendations section of the user interface 150 on the computing device 104.

By generating the high myopia determination 320 from a single vision screening and presenting it through the user interface 150, the vision screening device 102 and computing device 104 may enable early identification of patients at risk for developing high myopia. This approach may allow for timely implementation of preventive measures or treatments, potentially mitigating the progression of myopia in at-risk patients.

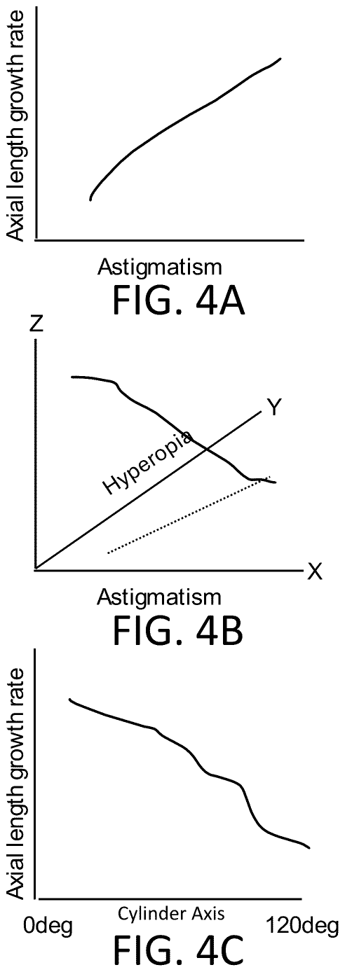

FIG. 4A illustrates a graph associating axial length growth rate with astigmatism, according to aspects of the present disclose. For example, the relationship between axial length growth rate and astigmatism may be complex and multifaceted. In examples, the axial length growth rate may increase as the degree of astigmatism increases. This relationship may not be linear, and the rate of increase may vary depending on the specific degree of astigmatism.

FIG. 4B illustrates a graph associating axial length growth rate, astigmatism, and hyperopia, according to aspects of the present disclose. For example, the presence of hyperopia may also influence the relationship between axial length growth rate and astigmatism. In some cases, when hyperopia is present, the axial length growth rate may be lower for a given degree of astigmatism compared to cases without hyperopia. The interaction between hyperopia and astigmatism may result in different patterns of axial length growth.

FIG. 4C illustrates a graph associating axial length growth rate and axis position of astigmatism, according to aspects of the present disclose. For example, the axis position of astigmatism may play a role in predicting the likelihood of high myopia development. In examples, the axis position may be classified as with-the-rule astigmatism or against-the-rule astigmatism. With-the-rule astigmatism may occur when the steepest meridian of the cornea is oriented vertically, while against-the-rule astigmatism may occur when the steepest meridian is oriented horizontally. The axial length growth rate may vary depending on the axis position of astigmatism. In examples, the axial length growth rate may be higher for against-the-rule astigmatism compared to with-the-rule astigmatism.

This difference in growth rates may be observed across various degrees of astigmatism. When predicting the likelihood of high myopia development, the trained model may take into account the axis position of astigmatism. In examples, the trained model may be biased to predict a lower likelihood of high myopia for patients with with-the-rule astigmatism. Conversely, the trained model may be biased to predict a higher likelihood of high myopia for patients with against-the-rule astigmatism.

The determination of the axis position of astigmatism may be an important factor in the prediction process. In examples, the process for predicting high myopia may determine the axis position of astigmatism indicating against-the-rule astigmatism. This determination may involve analyzing the orientation of the steepest meridian of the cornea and classifying it as horizontally oriented. Similarly, the vision screener may determine the axis position of astigmatism indicating with-the-rule astigmatism. This determination may involve identifying a vertically oriented steepest meridian of the cornea. The classification of astigmatism as either with-the-rule or against-the-rule may provide valuable information for the trained model to assess the likelihood of high myopia development.

With respect to the results shown in FIGS. 4A, 4B, and 4C, by incorporating these factors into the prediction process, the trained model may provide more accurate assessments of the likelihood of high myopia development. The consideration of astigmatism type, degree, and axis position, along with other relevant factors such as axial length growth rate and the presence of hyperopia, may contribute to a comprehensive evaluation of a patient's risk for developing high myopia.

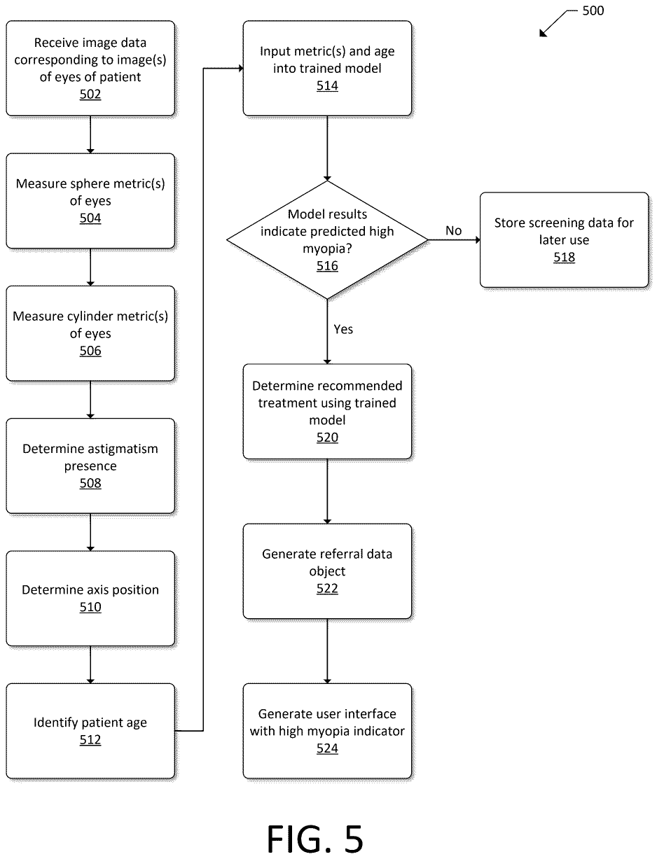

FIGS. 5-10 illustrate processes 500-1000 for user interface field configurability enhancements. The processes 500-1000 described herein are illustrated as collections of blocks in logical flow diagrams, which represent a sequence of operations, some or all of which may be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes 500-1000 are described with reference to the environments, architectures and systems described in the examples herein, such as, for example those described with respect to FIGS. 1-4, although the processes 500-1000 may be implemented in a wide variety of other environments, architectures and systems.

FIG. 5 illustrates a flowchart of a process 500 for retrieving several vision-related metrics and user-specific metrics to predict whether a patient will have high myopia at a later time, according to aspects of the present disclose. The order in which the operations or steps are described is not intended to be construed as a limitation, and any number of the described operations may be combined in any order and/or in parallel to implement process 500.

At step 502, the process 500 may include receiving, by a system such as the system described herein, image data corresponding to images of eyes of a patient. This step may include capturing images of the patient's eyes using one or more cameras integrated into the vision screening device. The cameras may utilize various imaging technologies, such as high-resolution digital sensors, infrared imaging, and/or multispectral imaging capabilities. In examples, the image data may be preprocessed within the vision screening device to enhance image quality, reduce noise, and/or extract relevant features. The received image data may include multiple views of each eye, potentially captured under different lighting conditions and/or at different focal lengths. In examples, the image data may be supplemented with metadata such as timestamps, camera settings, and/or patient identifiers. The vision screening device may temporarily store the received image data in local memory for immediate processing and/or sending to a connected computing device and/or server for further analysis. This step may also involve verifying the integrity and completeness of the received image data to ensure it is suitable for subsequent analysis steps in the vision screening process.

At step 504, the process 500 may include, by a system such as the system described herein, measuring sphere metrics of the eyes. This step may include analyzing the processed image data to determine the refractive power of the eye in different meridians. The measurement of sphere metrics may utilize various optical techniques, such as wavefront analysis and/or retinoscopy, to assess the overall refractive error of the eye. In examples, the vision screening device may project specific light patterns onto the retina and analyze the reflected light to calculate the sphere metrics. The measurement process may account for factors such as corneal curvature, lens shape, and axial length of the eye. The sphere metrics may be quantified in diopters, representing the degree of myopia or hyperopia present in the patient's eyes. In examples, the measurement may be performed separately for each eye, allowing for comparison between the left and right eyes. The vision screening device may employ advanced algorithms to interpret the reflected light patterns and convert them into precise sphere measurements. These measurements may serve as inputs for subsequent steps in the myopia prediction process.

At step 506, the process 500 may include, by a system such as the system described herein, measuring cylinder metrics of the eyes. This step may include analyzing the processed image data to determine the degree and axis of astigmatism present in the patient's eyes. The measurement of cylinder metrics may utilize advanced optical techniques, such as corneal topography, to assess the irregular curvature of the cornea or lens. In examples, the vision screening device may project a series of light patterns onto the retina and analyze the distortions in the reflected light to calculate the cylinder metrics. The measurement process may account for variations in corneal shape across different meridians of the eye. The cylinder metrics may be quantified in diopters and axis degrees, representing the magnitude and orientation of astigmatism. In examples, the measurement may be performed separately for each eye, allowing for comparison between the left and right eyes. The vision screening device may employ processes to interpret the reflected light patterns and convert them into precise cylinder measurements. These measurements may serve as inputs for subsequent steps in the astigmatism analysis and overall myopia prediction process, potentially influencing the assessment of axial length growth rates and the likelihood of high myopia development.

At step 508, the process 500 may include, by a system such as the system described herein, determining the presence of astigmatism in the patient's eyes. This step may utilize the cylinder metrics obtained in step 506 to assess whether astigmatism exists and to what degree. The vision screening device may employ processes to analyze the processed image data and cylinder measurements to detect irregularities in the corneal and/or lens curvature indicative of astigmatism. In examples, the device may compare the measured cylinder values against predefined thresholds to classify the presence and severity of astigmatism. The determination may be made separately for each eye, allowing for identification of unilateral or bilateral astigmatism. In cases where astigmatism is detected, the system may further categorize it as regular or irregular astigmatism based on the pattern of corneal irregularity. This step may also involve generating a visual representation of the astigmatism, such as a topographical map of the cornea, which may be stored for later reference or displayed on the user interface. The results of this step may serve as inputs for subsequent steps in the process, particularly in predicting the likelihood of high myopia development and determining appropriate treatment recommendations.

At step 510, the process 500 may include, by a system such as the system described herein, determining the axis position of astigmatism in the patient's eyes. This step may utilize the cylinder metrics obtained in step 506 and the astigmatism determination from step 508 to assess the orientation of the astigmatism. The vision screening device may employ advanced image processing techniques to analyze the processed image data and cylinder measurements to determine the axis of the steepest meridian of the cornea. In examples, the device may use vector analysis techniques to calculate the angle of the astigmatism axis. The axis position may be quantified in degrees, typically ranging from 0 to 180 degrees. The determination may be made separately for each eye, allowing for comparison between the left and right eyes. Based on the calculated axis position, the system may classify the astigmatism as with-the-rule (approximately 0 to 30 degrees), against-the-rule (approximately 60 to 120 degrees), or oblique (between 31-59 degrees). This classification may provide valuable input for subsequent steps in the process, particularly in predicting the likelihood of high myopia development and determining appropriate treatment recommendations.

At step 512, the process 500 may include, by a system such as the system described herein, identifying the age of the patient. This step may utilize the user profile data received earlier in the process and/or may involve querying a database containing patient information. The vision screening device may extract the patient's date of birth from the available data and calculate the current age at the time of the screening. In examples, the system may also identify the patient's age in months or days for more precise analysis, particularly for younger patients where small age differences may be significant.

At step 514, the process 500 may include, by a system such as the system described herein, inputting the collected metrics and age data into a trained model. This step may serve as a crucial integration point where various vision-related measurements and patient-specific information converge for analysis. The trained model, which may be a machine learning algorithm such as a neural network or decision tree, may receive inputs including the sphere metrics, cylinder metrics, astigmatism presence and type, axis position of astigmatism, and the patient's age. In examples, the model may also incorporate additional data points such as red reflex examination results, corneal topography maps, or historical vision screening data if available. The purpose of this step may be to process these diverse inputs through a sophisticated analytical framework, leveraging patterns and correlations identified during the model's training phase. The model may apply weightings to different input factors, potentially emphasizing certain metrics based on their predictive power for high myopia development. This step may involve complex calculations and data transformations within the model's architecture, potentially including normalization of input values, feature extraction, and/or dimensionality reduction techniques. The output of this step may be a preliminary assessment and/or risk score indicating the likelihood of the patient developing high myopia, which may then be used in subsequent steps for decision-making and recommendation generation.

At step 516, the process 500 may include, by a system such as the system described herein, evaluating the output from the trained model to determine if the results indicate predicted high myopia. The evaluation may involve comparing the model's output, which may be a probability score and/or risk assessment, to established criteria for high myopia prediction. In examples, the system may utilize multiple thresholds to categorize the risk level into different tiers, such as low, moderate, or high risk of developing high myopia. The decision-making process in this step may also take into account additional factors, such as family history of myopia and/or lifestyle factors, to refine the prediction accuracy. Depending on the outcome of this evaluation, the process may branch into different paths for subsequent actions. If the results indicate a high likelihood of developing high myopia, the system may trigger additional analysis or recommendation generation steps. Conversely, if the results do not meet the threshold for predicted high myopia, the process may proceed to step 518.

At step 518, the process 500 may include, by a system such as the system described herein, storing the screening data for later use. This step may serve as a data preservation and management phase, ensuring that valuable information collected during the vision screening process is retained for future reference and analysis. The vision screening device described herein may compile various data points collected throughout the screening, including the image data, sphere metrics, cylinder metrics, astigmatism determinations, and model outputs. In examples, the system may organize this data into a structured format, potentially creating a comprehensive patient record that includes all relevant measurements, predictions, and metadata. The storage process may involve encrypting sensitive patient information to maintain data security and privacy compliance. In examples, the system may implement data compression techniques to optimize storage efficiency while preserving data integrity. The stored data may be tagged with identifiers such as patient ID, screening date, and/or examiner information to facilitate easy retrieval and longitudinal analysis. This step may also involve synchronizing the stored data with a centralized database and/or cloud storage system, enabling access from multiple authorized devices or locations. By preserving this screening data, the system may support long-term patient monitoring, trend analysis, and continuous improvement of the predictive models used in high myopia risk assessment.

At step 520, the process 500 may include, by a system such as the system described herein, determining a recommended treatment using the trained model. This step may leverage the high myopia prediction generated in step 516 to suggest appropriate interventions. The trained model may analyze the patient's risk profile, including factors such as age, degree of myopia, astigmatism characteristics, and other relevant metrics, to recommend personalized treatment options. In examples, the model may consider a range of potential treatments, such as specialized contact lenses, atropine eye drops, increased outdoor time, or combinations thereof. The determination process may involve weighing the potential efficacy of different treatments against factors like the patient's age, lifestyle, and predicted myopia progression rate. In examples, the model may generate multiple treatment recommendations ranked by their predicted effectiveness. This step may also incorporate recent research findings and clinical guidelines to ensure up-to-date treatment suggestions. The output of this step may be a detailed treatment plan, potentially including dosage information for pharmaceutical interventions, schedules for wearing optical devices, and/or lifestyle modification recommendations. This treatment recommendation may then be presented to healthcare providers through the user interface for review and potential implementation in subsequent patient care steps.

At step 522, the process 500 may include, by a system such as the system described herein, generating a referral data object. This step may utilize the high myopia prediction and treatment recommendation generated in previous steps to create a comprehensive data package for healthcare providers. The referral data object may include key information such as the patient's demographic data, vision screening results, predicted likelihood of high myopia development, and/or recommended treatment options. In examples, the system may incorporate relevant images from the vision screening, such as corneal topography maps or retinal scans, into the referral data object. The generation process may involve aggregating data from various components of the vision screening device, including the reflection data analyzer, machine learning model, and treatment component. The referral data object may be structured in a standardized format to ensure compatibility with various electronic health record systems. In examples, the system may apply data encryption techniques to protect sensitive patient information during transmission. The referral data object may also include a unique identifier to facilitate tracking and follow-up care.

At step 524, the process 500 may include, by a system such as the system described herein, generating a user interface with a high myopia indicator. This step may serve as the culmination of the vision screening and analysis process, presenting the results in a clear and actionable format for healthcare providers and potentially patients. The user interface may display the predicted likelihood of high myopia development, potentially using color-coded indicators or numerical risk scores to convey the severity of the risk. In examples, the user interface may include graphical representations of the patient's current vision metrics, such as degree of myopia and astigmatism, alongside projections of potential vision deterioration over time. The high myopia indicator may be accompanied by explanatory text detailing the factors contributing to the prediction, which may help healthcare providers understand the basis for the assessment. In examples, the user interface may also present the recommended treatment options generated in step 520, potentially with interactive elements allowing providers to explore different treatment scenarios. The interface may incorporate data visualization techniques to illustrate complex relationships between various vision metrics and the predicted outcome. Additionally, the user interface may include options for generating reports, initiating referral processes, and/or scheduling follow-up examinations based on the screening results.

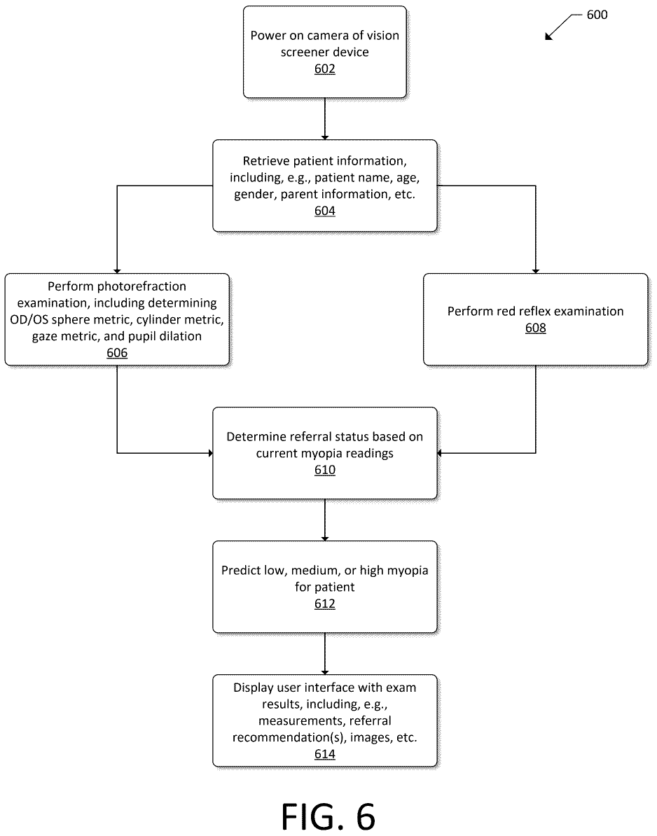

FIG. 6 illustrates a flowchart of a process 600 for use of a vision screening device to predict high myopia at a later time, according to aspects of the present disclose. The order in which the operations or steps are described is not intended to be construed as a limitation, and any number of the described operations may be combined in any order and/or in parallel to implement process 600.

At step 602, the process 600 may include, by a system such as the system described herein, powering on a camera of a vision screening device. This step may initiate the activation of various optical components within the device, including infrared emitters, image sensors, and focusing mechanisms. The camera activation process may include a self-diagnostic routine to ensure components are functioning correctly. In examples, the device may perform a calibration sequence to adjust for ambient lighting conditions. The camera activation may also trigger the initialization of image processing software and memory allocation for storing captured images. In examples, the device may display a visual indicator on its user interface to confirm that the camera is powered on and ready for use. The camera activation step may also involve establishing a connection with other components of the vision screening system, such as the main processor and/or external display devices. In examples, the device may load specific imaging profiles and/or settings based on the type of vision screening to be performed.

At step 604, the process 600 may include, by a system such as the system described herein, retrieving patient information. This step may include accessing and compiling various data points relevant to the patient's vision screening. The system may retrieve demographic information such as the patient's name, date of birth, gender, and unique identifier from a database or user input. In examples, the process may also gather more detailed information such as the patient's medical history, family history of vision conditions, and/or previous vision screening results. The retrieval process may involve querying multiple data sources, potentially including electronic health records, patient management systems, and/or cloud-based storage platforms. In examples, the system may also collect information about the patient's lifestyle factors that may influence vision health, such as screen time habits, outdoor activity levels, and/or dietary patterns. The process may incorporate data validation checks to ensure the accuracy and completeness of the retrieved information. In examples, the system may use natural language processing techniques to extract relevant information from unstructured text in medical notes and/or patient questionnaires. The retrieved patient information may be temporarily stored in the device's memory for use in subsequent steps of the vision screening process, potentially being used to customize the screening parameters or inform the analysis of screening results.

At step 606, the process 600 may include, by a system such as the system described herein, performing a photorefraction examination on the patient's eyes. This examination may utilize the vision screening device's light emitter to project specific light patterns onto the retina of a patient and capture the reflected light using the device's camera. The photorefraction process may determine several key metrics, including the OD/OS sphere measurement, which represents the degree of myopia or hyperopia in each eye. Additionally, the examination may assess the cylinder metric, indicating the presence and severity of astigmatism. The process may also measure gaze metrics to evaluate eye alignment and detect potential strabismus. Furthermore, the photorefraction examination may include pupil dilation measurements, which can provide insights into the patient's pupillary response and potentially indicate other ocular and/or neurological conditions. In examples, the vision screening device may capture multiple images and/or video sequences during the examination to ensure accurate measurements and allow for averaging of results.

At step 608, the process 600 may include, by a system such as the system described herein, performing a red reflex examination on the patient's eyes. This examination may utilize the vision screening device's light emitter to project a specific wavelength of light, typically in the red spectrum, onto the patient's retina and capture the reflected light using the device's camera. The red reflex examination may assess the transparency of the eye's optical media, including the cornea, aqueous humor, lens, and vitreous humor. In examples, the system may analyze the intensity, uniformity, and color of the reflected red light to detect potential abnormalities such as cataracts, retinal detachments, retinal tearing, and/or other ocular pathologies. The process may capture multiple images at different angles to provide a comprehensive assessment of the eye's internal structures. In examples, the red reflex examination may be performed concurrently with the photorefraction examination from step 606, allowing for efficient data collection and analysis. The results of the red reflex examination may be combined with other metrics to enhance the accuracy of the high myopia prediction model and provide a more comprehensive assessment of the patient's overall ocular health.

At step 610, the process 600 may include, by a system such as the system described herein, determining referral status based on current myopia readings. This step may utilize the data collected from the photorefraction examination in step 606 and the red reflex examination in step 608 to assess the patient's current myopia status. The system may compare the measured myopia levels against predefined thresholds, which may be based on age-specific guidelines and/or standardized referral criteria. In examples, the process may incorporate additional factors such as astigmatism measurements, gaze metrics, and pupil dilation data to provide a more comprehensive evaluation. The determination may involve applying decision-making processes that weigh multiple factors to assess whether the patient's condition warrants a referral to a specialist. In examples, the system may utilize machine learning models trained on large datasets of patient outcomes to enhance the accuracy of referral decisions. The referral status determination may also consider the rate of myopia progression if historical data is available, potentially flagging cases of rapid progression for urgent referral.

At step 612, the process 600 may include, by a system such as the system described herein, predicting whether the patient will have low, medium, or high myopia. This step may utilize the data collected and analyzed in previous steps, including the photorefraction examination results, red reflex examination results, and current myopia readings. The prediction process may employ the trained machine learning model to analyze the comprehensive set of vision metrics and patient data. In examples, the model may consider factors such as the degree of myopia, astigmatism measurements, axis position of astigmatism, patient age, and/or other relevant data points to generate a prediction. The prediction may be expressed as a probability and/or risk score for each category of myopia severity. In examples, the system may generate separate predictions for each eye, allowing for detection of potential asymmetry in myopia progression. The prediction step may also involve comparing the current assessment with historical data, if available, to evaluate the rate of myopia progression over time.

At step 614, the process 600 may include, by a system such as the system described herein, displaying a user interface with exam results, including measurements, referral recommendations, and/or images. This step may include presenting the collected and analyzed data in a comprehensive format for healthcare providers and potentially patients. The user interface may display key metrics such as the degree of myopia, astigmatism measurements, and gaze metrics obtained from the photorefraction examination. In examples, the interface may include visual representations of the red reflex examination results, potentially highlighting any detected abnormalities. The predicted myopia category (low, medium, or high) may be prominently displayed, possibly using color-coding or graphical indicators to convey the severity level. The interface may also present the referral status determination, providing guidance on whether the patient requires specialist consultation. In examples, the system may generate side-by-side comparisons of current results with historical data, if available, to illustrate myopia progression over time. The user interface may incorporate interactive elements allowing healthcare providers to explore detailed breakdowns of individual metrics or access raw examination data. Additionally, the interface may include options for generating printable reports, initiating electronic referrals, and/or scheduling follow-up examinations based at least in part on the screening results.

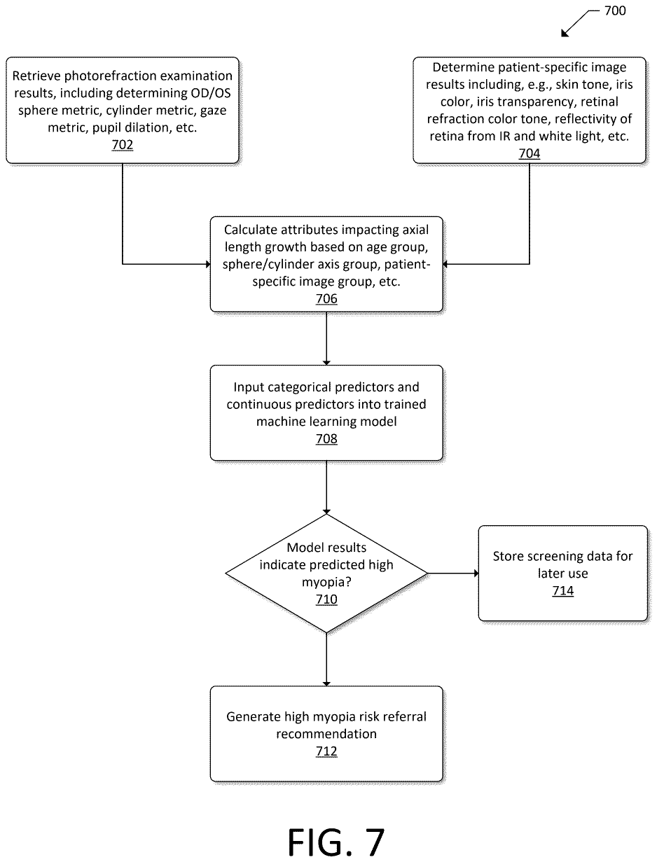

FIG. 7 illustrates a flowchart of a process 700 for use of a trained machine learning model to predict high myopia at a later time, according to aspects of the present disclose. The order in which the operations or steps are described is not intended to be construed as a limitation, and any number of the described operations may be combined in any order and/or in parallel to implement process 700.

At step 702, the process 700 may include, by a system such as the system described herein, retrieving photorefraction examination results for a patient. This process may begin with the system accessing stored data from a photorefraction examination. The retrieved results may include the OD/OS sphere metric, which represents the degree of myopia or hyperopia in each eye. Additionally, the system may extract the cylinder metric, indicating the presence and severity of astigmatism. The retrieval process may also include accessing gaze metrics that evaluate eye alignment and potential strabismus. Furthermore, the system may gather pupil dilation measurements obtained during the examination. In examples, the retrieval process may involve accessing multiple images or video sequences captured during the examination to ensure comprehensive data collection. The system may also perform initial data processing on the retrieved results, such as averaging multiple measurements or applying calibration factors. In examples where historical data is available, the system may retrieve previous examination results to enable comparison and trend analysis. The retrieved photorefraction examination results may then be formatted and prepared for input into the trained machine learning model in subsequent steps of the process.

At step 704, the process 700 may include, by a system such as the system described herein, determining patient-specific image results. The system may analyze images captured during the vision screening process to extract additional patient-specific data. This analysis may include determining the patient's skin tone, iris color, and/or iris transparency. The process may also involve assessing the retinal refraction color tone and measuring the reflectivity of the retina under both infrared IR and white light conditions. In examples, the system may utilize advanced image processing to accurately quantify these metrics. The determination of these patient-specific image results may involve multiple sub-processes, such as color analysis, pattern recognition, and reflectivity measurements. These metrics may provide valuable additional inputs for the machine learning model, potentially enhancing its ability to predict myopia progression. In examples, the system may compare the current image results with historical data, if available, to detect changes over time. The patient-specific image results determined in this step may be used in conjunction with the photorefraction examination results from step 702 to calculate attributes impacting axial length growth in subsequent steps of the process.

At step 706, the process 700 may include, by a system such as the system described herein, calculating attributes impacting axial length growth based at least in part on various patient-specific factors. This step may utilize the photorefraction examination results from step 702 and the patient-specific image results from step 704 to determine key attributes that may influence the growth of the eye's axial length. The calculation process may involve analyzing data across different categories, including age groups, sphere/cylinder axis groups, and patient-specific image groups. In examples, the system may employ statistical models and/or machine learning models to identify correlations between these factors and axial length growth patterns. The process may also consider additional variables such as genetic predisposition, environmental factors, and lifestyle habits that may impact eye growth. In examples, the system may generate a composite score and/or risk factor assessment based at least in part on the calculated attributes. This step may serve as a crucial intermediate stage, transforming raw examination data into meaningful predictors that can be used by the trained machine learning model in subsequent steps. The calculated attributes may provide a more nuanced understanding of the patient's risk for myopia progression, potentially improving the accuracy of the high myopia prediction in later steps of the process.

At step 708, the process 700 may include, by a system such as the system described herein, inputting categorical predictors and continuous predictors into the trained machine learning model. This step may serve as a crucial data integration point where various types of patient data are prepared and fed into the model for analysis. The categorical predictors may include discrete variables such as age groups, astigmatism types, and/or iris color categories determined in previous steps. Continuous predictors may encompass numerical measurements like the degree of myopia, cylinder metrics, and/or quantitative assessments of retinal reflectivity. In examples, the system may normalize and/or scale these predictors to ensure they are in a suitable format for the machine learning model. The input process may involve creating feature vectors that combine multiple predictors.

At step 710, the process 700 may include, by a system such as the system described herein, evaluating the output from the trained machine learning model to determine if the results indicate predicted high myopia. The evaluation may involve comparing the model's output, which may be a probability score and/or risk assessment, to established criteria for high myopia prediction. In examples, the system may utilize multiple thresholds to categorize the risk level into different tiers, such as low, moderate, or high risk of developing high myopia. This process may also take into account additional factors, such as family history of myopia or lifestyle factors to refine the prediction accuracy.

At step 712, the process 700 may include, by a system such as the system described herein, generating a high myopia risk referral recommendation. This step may utilize the output from the trained machine learning model evaluated in step 710 to determine if a referral is necessary. The system may generate a referral recommendation that includes the predicted likelihood of high myopia development, examination findings, and supporting data points that contributed to the prediction. In examples, the referral recommendation may be customized based at least in part on the severity of the predicted risk, potentially suggesting different specialists and/or follow-up timelines for varying risk levels. The system may incorporate standardized referral criteria to ensure the recommendations align with current best practices. The referral generation process may also consider factors such as the patient's age, current myopia progression rate, and/or additional risk factors identified during the examination. In examples, the system may generate a prioritized list of recommended interventions and/or treatments alongside the referral recommendation. This step may interface with the referral generator component described herein to create a structured referral data object that can be integrated into electronic health record systems and/or transmitted to other healthcare providers. The generated referral recommendation may serve as a crucial link between the predictive analysis performed by the machine learning model and the practical next steps in patient care management.

At step 714, the process 700 may include, by a system such as the system described herein, storing the screening data for later use. This step may serve as a data preservation and management phase, ensuring that valuable information collected during the vision screening process is retained for future reference and analysis. The system may compile various data points collected throughout the screening, including the photorefraction examination results, patient-specific image results, calculated attributes impacting axial length growth, and/or the output from the trained machine learning model. In examples, the system may organize this data into a structured format, potentially creating a comprehensive patient record that includes all relevant measurements, predictions, and metadata.

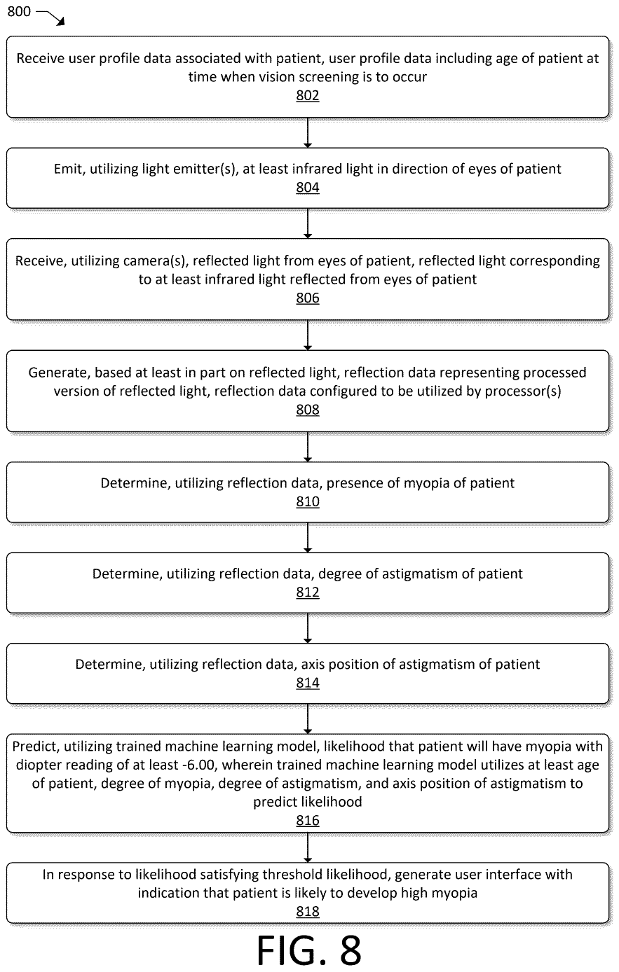

FIG. 8 illustrates a flowchart of a process 800 for implementing vision screening device enhancements, according to an embodiment. The order in which the operations or steps are described is not intended to be construed as a limitation, and any number of the described operations may be combined in any order and/or in parallel to implement process 800.

At step 802, the process 800 may include, by a system such as the system described herein, receiving user profile data associated with a patient, where the user profile data includes an age of the patient at a time when vision screen is to occur. For example, a data input interface may receive user profile data through various means, including manual entry by healthcare professionals, integration with electronic health record systems, and/or patient self-reporting via a secure portal. The process may include associating the incoming data with a unique patient identifier, ensuring proper linkage of information across multiple screenings or examinations. The user profile data may include diverse types of patient information, including demographic details, medical history, family vision health background, and lifestyle factors that may influence vision health.

At step 804, the process 800 may include, by a system such as the system described herein, emitting, utilizing the one or more light emitters, at least infrared light in a direction of eyes of the patient. This step may encompass a series of precise optical operations designed to illuminate the patient's eyes for examination. The system may activate one or more infrared light sources, which may be calibrated to emit light at specific wavelengths optimal for ocular examination. In examples, the process may involve adjusting the intensity and focus of the emitted light based at least in part on factors such as the patient's age, pupil size, or known eye conditions. The light emission may be pulsed or continuous, depending on the specific examination requirements.

At step 806, the process 800 may include, by a system such as the system described herein, receiving, utilizing the one or more cameras, reflected light from the eyes of the patient, where the reflected light corresponds to the at least infrared light reflected from the eyes of the patient. This step may involve capturing the light reflected from various structures within the patient's eyes using specialized optical sensors or cameras. The system may employ high-resolution image sensors capable of detecting subtle variations in light intensity and wavelength. In examples, the cameras may capture multiple images in rapid succession or record video sequences to account for eye movements or blinking. The received light data may be digitized and preprocessed to reduce noise and enhance relevant features.

At step 808, the process 800 may include, by a system such as the system described herein, generating, based at least in part on the reflected light, reflection data representing a processed version of the reflected light, the reflection data configured to be utilized by the one or more processors. This step may encompass a series of data processing operations designed to transform raw optical measurements into structured, analyzable information. The system may employ advanced signal processing techniques to filter and enhance the captured light data, potentially utilizing algorithms such as Fourier transforms and/or wavelet analysis to isolate relevant spectral components. In examples, the process may involve image reconstruction techniques to create detailed maps of the eye's structures from the reflected light patterns. The system may apply noise reduction methods to improve signal quality, potentially using adaptive filtering and/or machine learning-based approaches to distinguish meaningful data from artifacts.

At step 810, the process 800 may include, by a system such as the system described herein, determining, utilizing the reflection data, a degree of myopia of the patient. This step may encompass a detailed analysis of the processed reflection data to quantify the extent of myopia present in the patient's eyes. The system may employ advanced image processing techniques to analyze the curvature of the eye's optical components, particularly the cornea and lens, as represented in the reflection data. In examples, the process may involve comparing the patient's eye measurements against standardized models of eyes to calculate the degree of refractive error. The output of this step may be a numerical value representing the degree of myopia in diopters for each eye.

At step 812, the process 800 may include, by a system such as the system described herein, determining, utilizing the reflection data, a presence of astigmatism of the patient. This step may involve an analysis of the processed reflection data to quantify the extent and nature of astigmatism present in the patient's eyes. The system may employ image processing techniques to analyze the curvature of the cornea and lens across different meridians, as represented in the reflection data. In examples, the process may involve comparing the patient's corneal topography against idealized models to calculate the degree of irregular curvature. The output of this step may include numerical values representing the presence of astigmatism for each eye.

At step 814, the process 800 may include, by a system such as the system described herein, determining, utilizing the reflection data, an axis position of astigmatism of the patient. This step may involve a detailed analysis of the processed reflection data to identify and quantify the specific orientation of the astigmatism in the patient's eyes. The system may employ image processing techniques to analyze the patterns of light reflection across different meridians of the cornea and lens. In examples, the process may involve creating a topographical map of the eye's surface to precisely locate areas of irregular curvature. The system may utilize vector analysis techniques to calculate the angle of the steepest meridian, which may define the axis of astigmatism. This axis position may be quantified in degrees, typically ranging from 0 to 180 degrees. The determination may be made separately for each eye, allowing for comparison between the left and right eyes. Based on the calculated axis position, the system may classify the astigmatism as with-the-rule, against-the-rule, or oblique.

At step 816, the process 800 may include, by a system such as the system described herein, predicting, utilizing a trained machine learning model, a likelihood that the patient will have or otherwise develop myopia with a diopter reading of at least −6.00. This step may involve feeding the previously collected and processed data into a trained machine learning model for analysis. The machine learning model may utilize various input features, including at least the age of the patient, degree of myopia, presence of astigmatism, and axis position of astigmatism to generate the prediction. In examples, the model may incorporate additional factors such as red reflex examination results, skin tone, iris color, and iris transparency metrics to enhance prediction accuracy. The system may employ neural networks and/or ensemble methods to analyze complex relationships between these input features. The output of this step may be a probability score and/or risk assessment indicating the likelihood of the patient developing high myopia. In some implementations, the model may generate separate predictions for each eye, allowing for detection of potential asymmetry in myopia progression.

At step 818, the process 800 may include, by a system such as the system described herein, generating a user interface with an indication that the patient is likely to develop high myopia in response to the likelihood satisfying a threshold likelihood. This step may involve creating a visual representation of the prediction results for healthcare providers and potentially patients. The system may design the user interface to prominently display the high myopia risk assessment, potentially using color-coded indicators and/or numerical scores to convey the severity of the risk. In examples, the interface may include graphical representations of the patient's current vision metrics alongside projections of potential vision deterioration over time. The system may incorporate interactive elements allowing users to explore breakdowns of individual metrics or access raw examination data. Additionally, the interface may present the recommended treatment options and referral information described herein. In examples, the system may generate exportable data files from this interface, facilitating documentation and information sharing among healthcare providers.

Additionally, or alternatively, the process 800 may include determining an optimal treatment option for the patient from multiple potential treatment options. The system may utilize the trained machine learning models described herein to analyze the patient's data and predict the most effective intervention. Once a treatment option is selected, the system may display this recommendation via the user interface, providing healthcare providers with data-driven guidance for patient care. This functionality may enhance the clinical decision-making process by offering personalized treatment suggestions based on comprehensive analysis of the patient's vision metrics and risk factors.

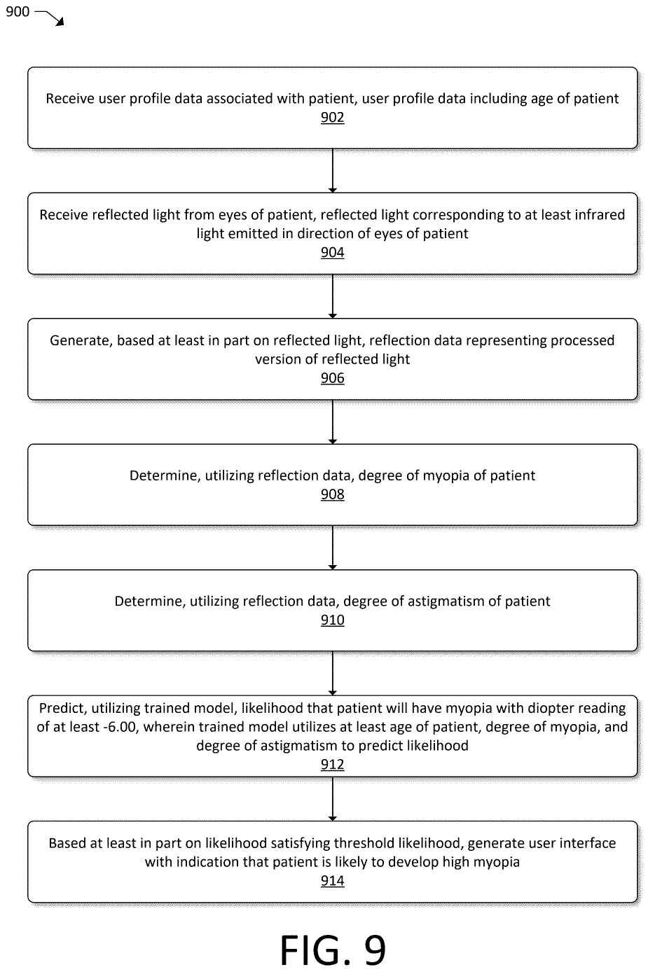

FIG. 9 illustrates a flowchart of another process 900 for implementing vision screening device enhancements, according to aspects of the present disclosure. The order in which the operations or steps are described is not intended to be construed as a limitation, and any number of the described operations may be combined in any order and/or in parallel to implement process 900.

At step 902, the process 900 may include, by a system such as the system described herein, receiving user profile data associated with a patient, where the user profile data includes an age of the patient at a time when vision screen is to occur. For example, a data input interface may receive user profile data through various means, including manual entry by healthcare professionals, integration with electronic health record systems, and/or patient self-reporting via a secure portal. The process may include associating the incoming data with a unique patient identifier, ensuring proper linkage of information across multiple screenings or examinations. The user profile data may include diverse types of patient information, including demographic details, medical history, family vision health background, and lifestyle factors that may influence vision health.

At step 904, the process 900 may include, by a system such as the system described herein, receiving, utilizing the one or more cameras, reflected light from the eyes of the patient, where the reflected light corresponds to the at least infrared light reflected from the eyes of the patient. This step may involve capturing the light reflected from various structures within the patient's eyes using specialized optical sensors or cameras. The system may employ high-resolution image sensors capable of detecting subtle variations in light intensity and wavelength. In examples, the cameras may capture multiple images in rapid succession or record video sequences to account for eye movements or blinking. The received light data may be digitized and preprocessed to reduce noise and enhance relevant features.

At step 906, the process 900 may include, by a system such as the system described herein, generating, based at least in part on the reflected light, reflection data representing a processed version of the reflected light, the reflection data configured to be utilized by the one or more processors. This step may encompass a series of data processing operations designed to transform raw optical measurements into structured, analyzable information. The system may employ advanced signal processing techniques to filter and enhance the captured light data, potentially utilizing algorithms such as Fourier transforms and/or wavelet analysis to isolate relevant spectral components. In examples, the process may involve image reconstruction techniques to create detailed maps of the eye's structures from the reflected light patterns. The system may apply noise reduction methods to improve signal quality, potentially using adaptive filtering and/or machine learning-based approaches to distinguish meaningful data from artifacts.

At step 908, the process 900 may include, by a system such as the system described herein, determining, utilizing the reflection data, a degree of myopia of the patient. This step may encompass a detailed analysis of the processed reflection data to quantify the extent of myopia present in the patient's eyes. The system may employ advanced image processing techniques to analyze the curvature of the eye's optical components, particularly the cornea and lens, as represented in the reflection data. In examples, the process may involve comparing the patient's eye measurements against standardized models of eyes to calculate the degree of refractive error. The output of this step may be a numerical value representing the degree of myopia in diopters for each eye.

At step 910, the process 900 may include, by a system such as the system described herein, determining, utilizing the reflection data, a presence of astigmatism of the patient. This step may involve an analysis of the processed reflection data to quantify the extent and nature of astigmatism present in the patient's eyes. The system may employ image processing techniques to analyze the curvature of the cornea and lens across different meridians, as represented in the reflection data. In examples, the process may involve comparing the patient's corneal topography against idealized models to calculate the degree of irregular curvature. The output of this step may include numerical values representing the presence of astigmatism for each eye.

At step 912, the process 900 may include, by a system such as the system described herein, predicting, utilizing a trained machine learning model, a likelihood that the patient will have or otherwise develop myopia with a diopter reading of at least −6.00. This step may involve feeding the previously collected and processed data into a trained machine learning model for analysis. The machine learning model may utilize various input features, including at least the age of the patient, degree of myopia, presence of astigmatism, and axis position of astigmatism to generate the prediction. In examples, the model may incorporate additional factors such as red reflex examination results, skin tone, iris color, and iris transparency metrics to enhance prediction accuracy. The system may employ neural networks and/or ensemble methods to analyze complex relationships between these input features. The output of this step may be a probability score and/or risk assessment indicating the likelihood of the patient developing high myopia. In some implementations, the model may generate separate predictions for each eye, allowing for detection of potential asymmetry in myopia progression.

At step 914, the process 900 may include, by a system such as the system described herein, generating a user interface with an indication that the patient is likely to develop high myopia based at least in part on the likelihood satisfying a threshold likelihood. This step may involve creating a visual representation of the prediction results for healthcare providers and potentially patients. The system may design the user interface to prominently display the high myopia risk assessment, potentially using color-coded indicators and/or numerical scores to convey the severity of the risk. In examples, the interface may include graphical representations of the patient's current vision metrics alongside projections of potential vision deterioration over time. The system may incorporate interactive elements allowing users to explore breakdowns of individual metrics or access raw examination data. Additionally, the interface may present the recommended treatment options and referral information described herein. In examples, the system may generate exportable data files from this interface, facilitating documentation and information sharing among healthcare providers.

Additionally, or alternatively, the process 900 may include determining an optimal treatment option for the patient from multiple potential treatment options. The system may utilize the trained machine learning models described herein to analyze the patient's data and predict the most effective intervention. Once a treatment option is selected, the system may display this recommendation via the user interface, providing healthcare providers with data-driven guidance for patient care. This functionality may enhance the clinical decision-making process by offering personalized treatment suggestions based on comprehensive analysis of the patient's vision metrics and risk factors.

Additionally, or alternatively, the process 900 may include determining the axis position of the patient's astigmatism utilizing the reflection data. In cases where this analysis indicates the presence of with-the-rule astigmatism, the system may adjust the parameters of its trained model. This adjustment may introduce a bias that reduces the likelihood of the model predicting high myopia for patients exhibiting this particular type of astigmatism. By incorporating this bias, the system may refine its predictive capabilities, potentially accounting for the observed correlation between with-the-rule astigmatism and lower rates of myopia progression in some patient populations.

Additionally, or alternatively, the process 900 may include determining the axis position of the patient's astigmatism utilizing the reflection data. In cases where this analysis indicates the presence of against-the-rule astigmatism, the system may adjust the parameters of its trained model. This adjustment may introduce a bias that increases the likelihood of the model predicting high myopia for patients exhibiting this particular type of astigmatism. By incorporating this bias, the system may refine its predictive capabilities, potentially accounting for the observed correlation between against-the-rule astigmatism and higher rates of myopia progression in some patient populations.

Additionally, or alternatively, the process 900 may include generating a machine learning model as described herein configured to predict likelihoods that patients will develop high myopia. Over a period of time, the system may receive feedback data indicating the accuracy of the predictions made by the machine learning model. This feedback data may be used to generate a comprehensive training dataset. The machine learning model may then undergo training utilizing this dataset, resulting in a trained machine learning model. The training process may involve making changes to the neural network architecture of the machine learning model, which may lead to increased accuracy in predicting the likelihoods of high myopia development. These modifications to the model may allow the model to better capture complex relationships between various input features and improve its predictive capabilities for different patient populations and scenarios.

Additionally, or alternatively, the process 900 may include determining that a patient should be referred to a healthcare professional associated with high myopia based at least in part on the likelihood of developing high myopia satisfying the threshold likelihood. In such examples, the system may generate a referral data object containing an indication of the patient's likelihood of developing high myopia, along with one or more images corresponding to reflected light from the patient's eyes and/or other aspects of a vision screening exam. Additionally, the process 900 may automatically generate a referral that incorporates the referral data object. This approach may streamline the referral process for patients identified as having a high risk of developing severe myopia, potentially enabling earlier intervention and more effective management of the condition.

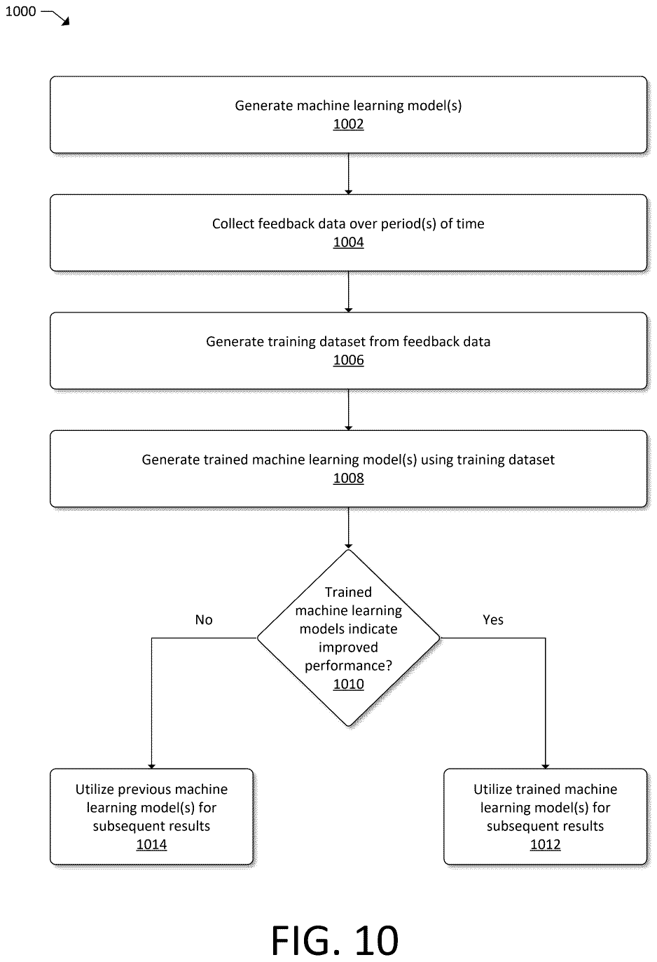

FIG. 10 is a flow diagram of an example process 1000 for the generation and training of artificial intelligence models (also referred to herein as machine learning models) to perform one or more of the processes described herein, according to an example described herein. The order in which the operations or steps are described is not intended to be construed as a limitation, and any number of the described operations may be combined in any order and/or in parallel to implement process 1000.

At step 1002, the process 1000 may include generating one or more artificial intelligence models, such as a machine learning model. A number of artificial intelligence techniques may be employed to generate and/or modify the layers and/or models described herein. Those techniques may include, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based artificial intelligence.

At step 1004, the process 1000 may include collecting feedback data over a period of time. The feedback data may include any data associated with determining questions and/or activities to present to a user, any described with respect to FIGS. 1-9, or any other data that may be utilized to perform the operations described herein. This information may include, for example, user input data, user activity data, etc.

At step 1006, the process 1000 may include generating a training dataset from the feedback data. Generation of the training dataset may include formatting the feedback data into input vectors for the artificial intelligence model to intake, as well as associating the various data with the outcomes of the questions and/or activities described herein.

At step 1008, the process 1000 may include generating one or more trained artificial intelligence models utilizing the training dataset. Generation of the trained artificial intelligence models may include updating parameters and/or weightings and/or thresholds utilized by the models to determine appropriate questions to present to the user, appropriate activities to recommend, and the like.

At step 1010, the process 1000 may include determining whether the trained artificial intelligence models indicate improved performance metrics. For example, a testing group may be generated where the outcomes of given questions and/or activities are known but not to the trained artificial intelligence models. The trained artificial intelligence models may generate results, which may be compared to the known results to determine whether the results of the trained artificial intelligence model produce a superior result than the results of the artificial intelligence model prior to training.

In examples where the trained artificial intelligence models indicate improved performance metrics, the process 1000 may include, at step 1012, utilizing the trained artificial intelligence models for generating subsequent results. For example, the trained artificial intelligence models may be utilized to determine appropriate questions to present to the user, appropriate activities to recommend, appropriate account balances to be maintained, and/or the like. The trained artificial intelligence models may be utilized in any scenario where models are utilized as described herein.

In examples where the trained artificial intelligence models do not indicate improved performance metrics, the process 1000 may include, at step 1014, utilizing the previous iteration of the artificial intelligence models for generating subsequent results.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

What is claimed is:

1. A method, comprising:
receiving user profile data associated with a patient, the user profile data including an age of the patient at a time when vision screening is to occur;
emitting, utilizing one or more light emitters, at least infrared light in a direction of eyes of the patient;
receiving, utilizing one or more cameras, reflected light from the eyes of the patient, the reflected light corresponding to the at least infrared light reflected from the eyes of the patient;
generating, based at least in part on the reflected light, reflection data representing a processed version of the reflected light, the reflection data configured to be utilized by one or more processors;
determining, utilizing the reflection data, a degree of myopia of the patient;
determining, utilizing the reflection data, a presence of astigmatism of the patient;
determining, utilizing the reflection data, an axis position of astigmatism of the patient;
predicting, utilizing a trained machine learning model, a likelihood that the patient will have myopia with a diopter reading of at least −6.00, wherein the trained machine learning model utilizes at least the age of the patient, the presence of myopia, the degree of astigmatism, the axis position of astigmatism, and patient gender to predict the likelihood; and
in response to the likelihood satisfying a threshold likelihood, generating a user interface with an indication that a likelihood of the patient developing high myopia is greater than a threshold likelihood, the user interface including a recommendation for referral based at least in part on the indication.

2. The method of claim 1, further comprising:
determining, utilizing the trained machine learning model, a treatment option to recommend for the patient from multiple potential treatment options; and
displaying the treatment option via the user interface.

3. The method of claim 1, further comprising performing, utilizing the one or more light emitters and the one or more cameras, a red reflex examination on the eyes of the patient such that red reflex examination results are determined, and wherein the trained machine learning model utilizes the red reflex examination results to predict the likelihood.

4. The method of claim 1, further comprising:
determining, utilizing image data generated by the one or more cameras:
a skin tone of the patient;
an iris color of the patient; and
an iris transparency metric of the patient; and
wherein the trained machine learning model utilizes at least one of the skin tone, the iris color, or the iris transparency metric to predict the likelihood.

5. A vision screening device, comprising:
one or more light emitters;
one or more cameras;
one or more processors; and non-transitory computer-readable media storing instructions that, when executed by the one or more processors, cause the vision screening device to perform operations comprising:
receiving user profile data associated with a patient, the user profile data including an age of the patient;
receiving reflected light from eyes of the patient, the reflected light corresponding to at least infrared light emitted in a direction of the eyes of the patient;
generating, based at least in part on the reflected light, reflection data representing a processed version of the reflected light;
determining, utilizing the reflection data, a degree of myopia of the patient;
determining, utilizing the reflection data, a degree of astigmatism of the patient;
predicting, utilizing a trained model, a likelihood that the patient will have myopia with a diopter reading of at least −6.00, wherein the trained model utilizes at least the age of the patient, the degree of myopia, and the degree of astigmatism to predict the likelihood; and
based at least in part on the likelihood satisfying a threshold likelihood, generating a user interface with an indication that a likelihood of the patient developing high myopia is greater than a threshold likelihood.

6. The vision screening device of claim 5, the operations further comprising:
determining, utilizing the trained model, a treatment option to recommend for the patient from multiple potential treatment options; and
causing the treatment option to be displayed via the user interface.

7. The vision screening device of claim 5, the operations further comprising performing a red reflex examination on the eyes of the patient such that red reflex examination results are determined, and wherein the trained model utilizes the red reflex examination results to predict the likelihood.

8. The vision screening device of claim 5, the operations further comprising:
determining, utilizing image data corresponding to an image of the patient:
a skin tone of the patient;
an iris color of the patient; and
an iris transparency metric of the patient; and
wherein the trained model utilizes the skin tone, the iris color, and the iris transparency metric to predict the likelihood.

9. The vision screening device of claim 5, the operations further comprising:
determining, utilizing the reflection data, an axis position of astigmatism of the patient, where the axis position indicates the patient has with-the-rule astigmatism; and
biasing the trained model such that predicting high myopia is less likely based at least in part on the patient having the with-the-rule astigmatism.

10. The vision screening device of claim 5, the operations further comprising:
determining, utilizing the reflection data, an axis position of astigmatism of the patient, where the axis position indicates the patient has against-the-rule astigmatism; and
biasing the trained model such that predicting high myopia is more likely based at least in part on the patient having the against-the-rule astigmatism.

11. The vision screening device of claim 5, wherein the trained model comprises a trained machine learning model, and the operations further comprise:
generating a machine learning model configured to predict likelihoods that patients will develop high myopia;
receiving feedback data over a period of time indicating accuracy of predicting likelihoods using the machine learning model;
generating a training dataset based at least in part on the feedback data; and
training the machine learning model utilizing the training dataset such that the trained machine learning model is generated, the trained machine learning model including changes to a neural network of the machine learning model that cause increased accuracy of predicting the likelihoods.

12. The vision screening device of claim 5, the operations further comprising:
determining, based at least in part on the likelihood satisfying the threshold likelihood, that the patient is to be referred to a healthcare profession associated with high myopia;
generating a referral data object including the indication that that the likelihood of the patient developing high myopia is greater than the threshold likelihood and including one or more images corresponding to the reflected light from the eyes of the patient; and
automatically generating a referral that includes the referral data object.

13. A system comprising:
one or more processors; and
non-transitory computer-readable media storing instructions that, when executed by the one or more processors, cause the system to perform operations comprising:
receiving data indicating an age of a patient;
generating, based at least in part on reflected light from eyes of the patient, reflection data representing a processed version of the reflected light;
determining, utilizing the reflection data, a degree of myopia of the patient;
determining, utilizing the reflection data, a degree of astigmatism of the patient;
predicting, utilizing a trained model, a likelihood that the patient will have myopia with a diopter reading of at least −6.00, wherein the trained model utilizes at least the age of the patient, the degree of myopia, and the degree of astigmatism to predict the likelihood; and
generating a user interface with an indication that a likelihood of the patient developing high myopia is greater than a threshold likelihood.

14. The system of claim 13, wherein the trained model comprises a machine learning model trained to predict likelihoods of high myopia from a single vision screening.

15. The system of claim 13, the operations further comprising determining myopia-related information from a parent of the patient, and wherein the trained model utilizes the myopia-related information from the parent of the patient to predict the likelihood.

16. The system of claim 13, wherein the degree of myopia comprises a first degree of myopia associated with a first eye of the patient, the degree of astigmatism comprises a first degree of astigmatism associated with the first eye of the patient, the likelihood comprises a first likelihood that the first eye of the patient will be associated with high myopia, and the operations further comprise:
determining, utilizing the reflection data, a second degree of myopia associated with a second eye of the patient;
determining, utilizing the reflection data, a second degree of astigmatism associated with a second eye of the patient; and
predicting, utilizing the trained model, a second likelihood that the patient will have myopia in the second eye with a diopter reading of at least −6.00, wherein the trained model utilizes at least the age of the patient, the second degree of myopia, and the second degree of astigmatism to predict the second likelihood.

17. The system of claim 13, the operations further comprising:
determining, utilizing the trained model, a treatment option to recommend for the patient from multiple potential treatment options; and
displaying the treatment option via the user interface.

18. The system of claim 13, the operations further comprising performing a red reflex examination on the eyes of the patient such that red reflex examination results are determined, and wherein the trained model utilizes the red reflex examination results to predict the likelihood.

19. The system of claim 13, the operations further comprising:
determining, utilizing image data corresponding to an image of the patient:
a skin tone of the patient;
an iris color of the patient; and
an iris transparency metric of the patient; and
wherein the trained model utilizes the skin tone, the iris color, and the iris transparency metric to predict the likelihood.

20. The system of claim 13, wherein the trained model comprises a trained machine learning model, and the operations further comprise:
generating a machine learning model configured to predict likelihoods that patients will develop high myopia;
receiving feedback data over a period of time indicating accuracy of predicting likelihoods using the machine learning model;
generating a training dataset based at least in part on the feedback data; and training the machine learning model utilizing the training dataset such that the trained machine learning model is generated, the trained machine learning model including changes to a neural network of the machine learning model that cause increased accuracy of predicting the likelihoods.
